(19) United States
(12) Patent Application Publication
(10) Pub. No.: US 2002/0068761 A1
(43) Pub. Date: Jun. 6, 2002

(54) Gallium complexes of 3-hydroxy-4-pyrones to treat mycobacterial infections

(76) Inventor: Lawrence R. Bernstein, Menlo Park, CA (US)

Correspondence Address: REED & ASSOCIATES, 800 MENLO AVENUE, SUITE 210, MENLO PARK, CA, 94025, US

(21) Appl. No.: 10/032,126

(22) Filed: Dec. 20, 2001

Related U.S. Application Data
(63) Continuation-in-part of application No. 09/684,684, filed on Oct. 4, 2000.
(60) Provisional application No. 60/157,460, filed on Oct. 4, 1999.

(51) Int. Cl. A61K 31/35; A61K 31/555

(52) U.S. Cl. 514/460; 514/184

(57) ABSTRACT

Methods are provided for the use of gallium complexes of 3-hydroxy-4-pyrones in the treatment or prevention of infections caused by a prokaryote of the genus Mycobacterium, including but not limited to those infections due to M. tuberculosis and M leprae. Methods are also provided for the treatment of immunocompromised patients infected by these and other mycobacteria species, including species (such as M. avium, M. aurum, and M. smegmatis) that are not pathogenic to immunocompetent individuals but may cause disease in immunocompromised patients.

### CROSS-REFERENCE TO RELATED APPLICATIONS

This is a continuation-in-part of U.S. patent application Ser. No. 09/684,684, filed Oct. 4, 2000, which claims priority to U.S. Provisional Patent Application Serial No. 60/157,460, filed Oct. 4, 1999, the disclosures of which are incorporated by reference herein.

### TECHNICAL FIELD

The present invention relates generally to the treatment or prevention of intracellular infections. More particularly, the invention relates to the treatment or prevention of intracellular infections by bacteria of the genus Mycobacterium, such as Mycobacterium tuberculosis (which causes tuberculosis) and Mycobacterium leprae (which causes leprosy), including strains that are resistant to conventional antimicrobials. The invention additionally relates to the treatment of immunocompromised patients infected by these and other mycobacteria species, including species (such as Mycobacterium avium, Mycobacterium aurum, and Mycobacterium smegmatis) that are not pathogenic to immunocompetent individuals but may cause disease in immunocompromised patients.

### BACKGROUND

Intracellular bacteria of the genus Mycobacterium are responsible for several serious and debilitating diseases, including tuberculosis and leprosy. Tuberculosis is a contagious bacterial infection caused by M. tuberculosis that afflicts millions of people worldwide. Current treatment regimens involve the daily administration of three or four antibiotics (e.g., combinations of rifampin, isoniazid, streptomycin, ethambutol, and pyrazinamide) for six to twelve months. Such regimens commonly produce significant adverse effects and low patient compliance. The low patient compliance in turn often leads to the development of drug-resistant strains. There is therefore a need for an improved method of treating patients suffering from tuberculosis that involves a simpler and shorter dosing regimen using a low-toxicity agent; such a regimen should lead to high patient compliance and a low incidence of emergent drug resistance.

Leprosy, a debilitating and disfiguring disease that affects skin and nerve tissue, is caused by M. leprae. The current treatment for leprosy resembles tuberculosis combination therapy, comprising administration of three or four antibiotics (e.g., combinations of dapsone, rifampin, rifampicine, and clofazimine). Due to the complexity of the current dosing regimen and its high expense, patient compliance tends to be low, leading to the emergence of drug-resistant strains. These problems, as with tuberculosis, are compounded by the high concentration of leprosy in developing countries, where medical supplies and education are commonly in short supply.

Although mycobacterial species pose serious threats to healthy individuals, the threats are greater for immunocompromised patients. These patients, such as those infected by HIV, are highly susceptible to mycobacterial infections and are less responsive to conventional combination anti-mycobacterial therapies. Therefore, there is a need for a more effective method of treating mycobacterial infections in immunocompromised patients. Further, an anti-mycobacterial agent must be compatible with ongoing therapies in these patients.

Gallium is known to prevent the replication of intracellular pathogens, including mycobacteria (Olakanmi et al., 1997, “Gallium inhibits growth of pathogenic mycobacteria in human macrophages by disruption of bacterial iron metabolism: a new therapy for tuberculosis and Mycobacterium avium complex?”, J Invest Med 45:234A). Without in any way restricting the invention to a particular mechanism of action, it is thought that gallium exerts its antibacterial activity through a novel mechanism: interference with bacterial iron uptake and metabolism through mimicry of ferric iron. Replicating bacterial cells (as other replicating cells) have a high iron requirement, due mainly to their need to produce ribonucleotide reductase (RR), a ferric iron-bearing enzyme essential for the synthesis of DNA. Gallium is chemically very similar to ferric iron, and so can be mistakenly taken up by these cells and incorporated into RR instead of iron. As gallium-containing RR (or iron-free RR in general) is nonfunctional, DNA cannot be synthesized and the affected cell attempting to replicate will ultimately undergo apoptosis.

In the case of tuberculosis, infecting mycobacteria live primarily within macrophages, making the bacteria particularly hard to reach and to treat with most antibacterial compounds. Macrophages, however, particularly those that are infected, naturally take up large amounts of iron by overexpressing transferrin receptor, which binds to the iron transport protein transferrin. Gallium administered orally as gallium maltolate binds to transferrin in place of iron, and so can gain entry into the macrophages and be taken up by the infecting mycobacteria. It is also possible that gallium administered as gallium maltolate can be taken up by macrophages and other target tissues by non-transferrin dependent mechanisms. The therapeutic mechanisms of action for gallium are discussed by Bernstein (1998), “Mechanisms of therapeutic activity for gallium”, Pharmacol Rev 50:665-682.

Gallium nitrate has been administered to humans by intravenous infusion, though with significant potential side effects such as nephrotoxicity. This is because a significant fraction of gallium from intravenous gallium nitrate circulates as the gallate radical (Ga(OH)₄⁻). Gallate, as a small charged molecule, is rapidly excreted in the urine and can transiently reach high concentrations in the kidney, where it can react to form precipitates (see Webster et al. (1999), “A pharmacokinetic and phase II study of gallium nitrate in patients with non-small cell lung cancer”, Cancer Chemother Pharmacol 45: 55-58). Free gallium is also unavailable for target cell uptake by transferrin-dependent mechanisms. In addition, compliance for a drug requiring slow intravenous infusion, such as intravenous gallium nitrate, can be problematic over the several months required for conventional anti-mycobacterial therapy. By contrast, orally administered gallium maltolate provides a novel, safer, and potentially more effective alternative to the anti-mycobacterial agents currently in use or to gallium salts such as gallium nitrate that require intravenous administration. Due its unique mechanism of action, and the likely synergy of gallium with other antibiotics, oral gallium maltolate should also significantly shorten the course of treatment for mycobacterial infections. Thus, administration of gallium maltolate provides an improved method of treating patients suffering from mycobacterial infections, such as tuberculosis and leprosy, by providing a simpler as well as a shorter dosing regimen.

### SUMMARY OF THE INVENTION

One aspect of the invention relates to a method of treating a patient infected by a prokaryote of the genus Mycobacterium by administering a therapeutically effective amount of a neutral 3:1 gallium complex of a 3-hydroxy-4-pyrone.

Yet another aspect of the invention relates to an improved method of treating a patient infected by a prokaryote of the genus Mycobacterium by administering to the patient a combination of antimicrobial agents selected from the group consisting of amikacin, aminosalicylic acid, azithromycin, capreomycin, ciprofloxacin, clarithromycin, clofazimine, cycloserine, dapsone, erythromycin, ethambutol, ethionamide, isoniazid, kanamycin, minocycline, ofloxacin, protionamide, pyrazinamide, rifabutin, rifampicine, rifampin, sparfloxacin, streptomycin, trimethoprim sulfamethoxazole, tobramycin, and viomycin; the improvement comprising administering a therapeutically effective amount of a neutral 3:1 gallium complex of a 3-hydroxy-4-pyrone.

Another aspect of the invention pertains to a method of treating an immunocompromised patient infected by a prokaryote of the genus Mycobacterium by administering a therapeutically effective amount of a neutral 3:1 gallium complex of a 3-hydroxy-4-pyrone.

Yet another aspect of the invention relates to a method of preventing infection by a prokaryote of the genus Mycobacterium by administering a prophylactically effective amount of a neutral 3:1 gallium complex of a 3-hydroxy-4-pyrone.

### DETAILED DESCRIPTION OF THE INVENTION

As noted above, the present invention is directed to methods for treating and preventing infection by pathogenic intracellular prokaryotes of the genus Mycobacterium using a neutral 3:1 gallium complex of a 3-hydroxy-4-pyrone.

Examples of mycobacterial species to which the methods of the invention find utility include, by way of illustration and not limitation, M. leprae and M. tuberculosis.

Prior to discussing this invention in further detail, the following terms will be defined. Unless defined below, the terms used herein have their normally accepted meanings.

### DEFINITIONS

As used herein, the following terms have the definitions given below:

The term “genus Mycobacterium” is intended to include all members of that genus including strains that are resistant to conventional antimicrobials. The term is intended to encompass species such as M. tuberculosis and M. leprae, as well as species such as M. avium, M. aurum, and M. smegmatis, which are not typically pathogenic to healthy or immunocompetent individuals but that may cause disease in immunocompromised patients. In general, the mycobacterial species of interest include, by way of illustration and not limitation, M. africanum, M. aurum, M. avium, M. avium paratuberculosis, M. balnei, M. bovis, M. chelonae, M. genitalium, M. gallisepticum, M. gastri, M. goodii, M. gordonae, M. haemophilum, M. intracellulare, M. kansasii, M. leprae, M. lepraemurium, M. malmoense, M. microti, M. penetrans, M. platypoecilus (commonly known as M. marinarum), M. pneumoniae, M. scrofulvaeum, M. simiae, M. smegmatis, M. szulgai, M. terrae-trivial, M. tuberculosis, M. ulcerans, and M. xenopi.

The term “neutral 3:1 gallium complex of a 3-hydroxy-4-pyrone” refers to an electrostatically neutral complex of Ga³⁺ (Ga(III)) and three equivalents of the anionic form of a 3-hydroxy-4-pyrone, which complex is represented by the formula [Ga³+(py⁻)₃], wherein py⁻ represents the anionic form of a 3-hydroxy-4-pyrone as defined below. Because such complexes do not dissociate to any significant extent in aqueous solutions maintained at a pH of from about 5 to about 9, these complexes remain predominantly electrostatically neutral in such solutions.

The term “3-hydroxy-4-pyrone” refers to a compound of Formula 1:

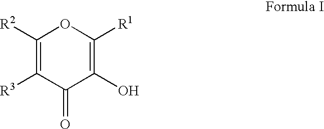

wherein R¹, R², and R³ are independently selected from the group consisting of hydrogen and a —C₁₋₆ alkyl group. The —C₁₋₆ alkyl group can be branched or unbranched but is preferably unbranched. Suitable —C₁₋₆ alkyl groups include, by way of illustration and not limitation, methyl, ethyl, isopropyl, and n-propyl. Preferred —C₁₋₆ alkyl groups are those having 1-3 carbons, in particular, methyl, and ethyl. Single substitution is preferred, particularly substitution at the 2or the 6-position, with substitution at the 2-position being most preferred.

Exemplary compounds encompassed by the term “a 3-hydroxy-4-pyrone” are described below.

The unsubstituted form of Formula 1 (R¹, R², and R³ are H) is known as pyromeconic acid.

Compounds of Formula 1 where R² and R³ are H include: 3-hydroxy-2-methyl-4-pyrone (R¹ is —CH₃), which is also known as maltol or larixinic acid; and 3-hydroxy-2-ethyl-4-pyrone (R¹ is —C₂H₅), which is sometimes referred to as ethyl maltol or ethylpyromeconic acid. Both of these are preferred for use in the methods of the invention, in particular 3-hydroxy-2-methyl-4-pyrone.

Compounds of Formula 1 where R¹ and R³ are H include 3-hydroxy-6-methyl-4-pyrone (R² is —CH₃).

The term “an anion of a 3-hydroxy-4-pyrone” refers to a compound defined in Formula 1 above wherein the hydroxyl proton has been removed so as to provide for the anionically charged form of the compound.

The term “administering” is intended to refer to the oral administration of any conventional form for the oral delivery of a pharmaceutical composition to a patient (e.g., human or other mammal) that results in the deposition of the pharmaceutical composition into the gastrointestinal tract (including the gastric portion of the gastrointestinal tract, i.e., the stomach) of the patient.

By the term “therapeutically effective” amount of a drug is meant a nontoxic but sufficient amount of a compound to provide the desired effect at a reasonable benefit/risk ratio. The desired effect may be alleviation of the signs, symptoms, or causes of a disease, or any other desired alteration of a biological system. In particular, a therapeutically effective amount refers to an amount of gallium complex administered such that a blood plasma gallium concentration is obtained that is sufficient to enable treatment or prevention of the infection of interest. The therapeutically effective amount necessary to prevent a disease is referred to as the “prophylactically effective amount”.

The term “therapeutic agent” refers to any additional therapeutic agent that is co-administered with the neutral 3:1 gallium complex of a 3-hydroxy-4-pyrone in the methods of the invention. The additional therapeutic agent can be administered by any route or in any dosage form. Co-administration can be by simultaneous or subsequent administration. Simultaneous administration can be in the form of separate or combined dosage forms, with the caveat that a combined dosage form should be suited for oral administration since that is the preferred route of delivery for the neutral 3:1 gallium complex of a 3-hydroxy-4-pyrone.

The term “treat,” as in to “treat” a condition, is intended to include (1) preventing the condition, i.e., avoiding any clinical symptoms of the condition, (2) inhibiting the condition, that is, arresting the development or progression of clinical symptoms, and/or (3) relieving the condition, i.e., causing regression of clinical symptoms.

The term “patient”, as in “treatment of a patient”, is intended to refer to an individual animal or human afflicted with or prone to a condition, disorder, or disease as specified herein, and typically refers to mammals, particularly humans.

By “pharmaceutically acceptable” is meant a material that is not biologically or otherwise undesirable, i.e., the material may be administered to an individual along with the neutral 3:1 gallium complex of a 3-hydroxy-4-pyrone (and any additional therapeutic agents) without causing any undesirable biological effects or interacting in a deleterious manner with any of the other components of the pharmaceutical composition in which it is contained.

“Optional” or “optionally” means that the subsequently described circumstance may or may not occur, so that the description includes instances where the circumstance occurs and instances where it does not. For example, recitation of an additive as “optionally present” in a formulation herein encompasses both the formulation containing the additive and the formulation not containing the additive.

The term “immunocompromised patient” is intended to refer to a patient suffering from an immunodeficiency. This includes a patient who has an autoimmune disease such as systemic lupus erythematosus or rheumatoid arthritis, a patient who is infected with a retrovirus, a patient who is undergoing chemotherapy, a patient with a genetic mutation that predisposes him or her to an immunodeficient state, or a patient who is a transplant recipient taking anti-rejection medications. Retroviruses that may be the causative agent in producing an immunodeficiency in a patient include but are not limited to, the human spumavirus, Mason-Pfizer monkey bovine leukaemia virus, mouse mammary tumor virus, avian leukosis virus, murine leukemia virus, Rous sarcoma virus, feline leukemia virus, feline immunodeficiency virus, simian immunodeficiency virus, human T cell leukemia viral species (“HTLV1”, “HTLV2”), and human immunodeficiency virus (“HIV”). Of particular interest is HIV, which refers to one or more members of the group of retroviruses that are members of the primate lentivirus group of the genus Lentiviridae and are capable of infecting a human, whether or not this capability has been demonstrated. For example, HIV-1 and HIV-2 are examples of primate lentiviruses that are known to infect humans. Infection of a human by a lentivirus that is not named and differs from all known HIV strains is also considered to be within the scope of the invention.

It must be noted that as used herein and in the claims, the singular forms “a”, “and”, and “the” include reference to both the singular and plural unless the context clearly dictates otherwise. Thus, for example, reference to “a therapeutic agent” in a formulation includes two or more active agents, reference to “a carrier” includes two or more carriers, and so forth.

### Synthesis And Methodology

The neutral 3:1 gallium complex of a 3-hydroxy-4-pyrone useful in the methods of the present invention can be easily synthesized by methods that are well known in the art. U.S. Pat. No. 6,004,951 to Bernstein describes the synthesis of neutral 3:1 gallium complexes of the compounds of Formula 1, and is incorporated herein by reference. In general, the complexes are synthesized by reacting the desired 3-hydroxy-4-pyrone with gallium ions in solution. The gallium ions can be derived from a gallium salt, such as a gallium halide or gallium nitrate compound. The 3-hydroxy-4-pyrone starting materials either occur naturally or may be obtained commercially or by known synthetic methods. Typical solvents include water, ethanol, methanol, and chloroform. The hydroxypyrone and the gallium ions are mixed in 3:1 molar proportions, preferably with a slight excess of hydroxypyrone to insure a complete reaction of all the gallium.

### Pharmaceutical Compositions and Modes of Administration

The methods of this invention are achieved by using a pharmaceutical composition comprising a neutral 3:1 gallium complex of a 3-hydroxy-4-pyrone. Preferred complexes include, by way of illustration and not limitation, the 3:1 complex of maltol with gallium, which is referred to as tris(3-hydroxy-2-methyl-4H-pyran-4-onato)gallium or gallium maltolate; and the 3:1 complex of ethyl maltol with gallium, referred to as tris(3-hydroxy-2-ethyl-4H-pyran-4-onato)gallium or gallium ethyl maltolate.

The compounds may be administered orally, parenterally (including by subcutaneous, intravenous, and intramuscular injection), transdermally, rectally, nasally, opthalmically, buccally, sublingually, topically, vaginally, etc., in dosage formulations containing one or more conventional nontoxic pharmaceutically acceptable carriers. For example, topical application to cutaneous lesions, such as the lesions caused by leprosy, is contemplated. The typical delivery route, however, is oral.

Depending on the intended mode of administration, the pharmaceutical compositions may be in the form of solid, semi-solid, or liquid dosage forms, such as, for example, tablets, suppositories, pills, capsules, powders, liquids, suspensions, creams, ointments, lotions, or the like, preferably in unit dosage form suitable for single administration of a precise dosage. The compositions contain an effective amount of the neutral 3:1 gallium complex of a 3-hydroxy-4-pyrone, generally although not necessarily in combination with a pharmaceutically acceptable carrier and, in addition, may include other pharmaceutical agents, adjuvants, diluents, buffers, etc. Various dosage forms of neutral 3:1 gallium complexes of a 3-hydroxy-4-pyrone suitable for use in the methods of the instant invention are set forth by Bernstein, U.S. Pat. No. 6,004,951.

As noted above, preferred compositions herein are oral formulations, which include delayed release oral formulations. While the neutral 3:1 complex of gallium with 3-hydroxy-4-pyrones delivers gallium to the bloodstream from the gastrointestinal tract, partial dissociation may occur of the neutral 3:1 complex of gallium with 3-hydroxy-4-pyrone under acidic conditions (generally at a pH of about 4 or less). Such acidic conditions may be present in the stomach. The dissociation may result in formation of less absorbable complexes, together with free hydroxypyrone and ionic gallium. Accordingly, in order to maintain the orally delivered gallium in a form that is highly absorbable in the gastrointestinal tract, the pharmaceutical compositions of this invention may be formulated to contain a means to inhibit dissociation of this complex when exposed to the acidic conditions of the stomach. Means to inhibit or prevent dissociation of this complex when exposed to the acidic conditions of the stomach are described in detail by Bernstein, U.S. Pat. No. 6,004,951. Suitable compositions can include a buffering agent that is effective to shift the equilibrium towards the neutral 3:1 complex within a mixture of gallium hydroxypyrone complexes (including the 1:1, 2:1, and 3:1 complexes), which may result when the composition reaches acidic conditions in the stomach of the individual. Another means of inhibiting or preventing dissociation is to encapsulate the pharmaceutical composition in a material that does not dissolve until the small intestine of the individual is reached, such as with enteric coated tablets, granules, or capsules, as is well known in the art.

### Methods of Pharmaceutical Treatment

In general, the therapeutic plasma levels of gallium are approximately 1 to 5,000 ng/mL, particularly approximately 100 to 1500 ng/mL. Oral doses to achieve these therapeutic levels are approximately 10 to 2,500 mg of the complex per day, particularly approximately 100 to 750 mg per day. The complex is preferably administered in single dose form, but may be administered in multiple doses per day. The complex also is preferably administered at least one hour before meals and at least two hours after meals, but other schedules are also acceptable.

Treatment of Mycobacterial Infection

One embodiment of the invention involves treating a patient infected by a prokaryote of the genus Mycobacterium by administering to the patient a therapeutically effective amount of a neutral 3:1 gallium complex of a 3-hydroxy-4-pyrone. The neutral 3:1 (hydroxypyrone:gallium) complex is one in which the 3-hydroxy-4-pyrone is selected from the group of compounds represented by Formula 1, as defined above. The therapeutically effective amount is such that a blood plasma gallium concentration is achieved that is sufficient to enable beneficial treatment of the infection.

Of particular interest is treatment of those patients that have been infected with Mycobacterium tuberculosis or Mycobacterium leprae.

In an exemplary dosing regimen, a patient infected by a prokaryote of the genus Mycobacterium preferably will be given about 100 to 750 mg/day of the complex for about 30 to 365 days or longer, the actual duration of therapy being determined by the specific infection to be eradicated. Another exemplary dosing regimen, for example for treatment of M. tuberculosis includes administration of 150 to 750 mg/day for about 30 to 180 days. Yet another exemplary dosing regimen, for example for treatment of M. leprae includes administration of 150-750 mg/day for about 30 to 365 days.

Optionally, it may be desired to include additional therapeutic agents with the neutral 3:1 gallium complex of a 3-hydroxy-4-pyrone. Such additional agents include, by way of example and not limitation, one or more antimicrobial agents. Exemplary antimicrobial agents are those that have a known efficacy against one or more mycobacteria species. These include amikacin, aminosalicylic acid, azithromycin, capreomycin, ciprofloxacin, clarithromycin, clofazimine, cycloserine, dapsone, erythromycin, ethambutol, ethionamide, isoniazid, kanamycin, minocycline, ofloxacin, protionamide, pyrazinamide, rifabutin, rifampicine, rifampin, sparfloxacin, streptomycin, trimethoprim sulfamethoxazole, tobramycin, and viomycin, and combinations thereof. Particularly preferred are combinations of one or more agents selected from the group consisting of ethambutol, isoniazid, pyrazinamide, rifampin, and streptomycin. The term “antimicrobial agent” is intended to include the compounds identified above, as well as their pharmaceutically acceptable isomers, salts, hydrates, solvates, esters, and prodrug derivatives, e.g., isoniazid hydrazide.

Treatment of mycobacterial infection in combination with existing therapies

Another embodiment of the invention involves an improved method of treating a patient infected by a prokaryote of the genus Mycobacterium by administering to the patient a combination of antimicrobial agents selected from the group consisting of amikacin, aminosalicylic acid, azithromycin, capreomycin, ciprofloxacin, clarithromycin, clofazimine, cycloserine, dapsone, erythromycin, ethambutol, ethionamide, isoniazid, kanamycin, minocycline, ofloxacin, protionamide, pyrazinamide, rifabutin, rifampicine, rifampin, sparfloxacin, streptomycin, trimethoprim sulfamethoxazole, tobramycin, and viomycin; the improvement comprising administering a therapeutically effective amount of a neutral 3:1 gallium complex of a 3-hydroxy-4-pyrone. The neutral 3:1 (hydroxypyrone:gallium) complex is one in which the hydroxypyrone is selected from the group of compounds represented by Formula 1, as defined above. The therapeutically effective amount is that which achieves a blood plasma gallium concentration that is sufficient to facilitate the antimicrobial combination therapy.

Of particular interest is treatment of those patients that have been infected with Mycobacterium tuberculosis or Mycobacterium leprae.

Particularly preferred for co-administration with the gallium hydroxypyrone complex are combinations of one or more agents selected from the group consisting of ethambutol, isoniazid, pyrazinamide, rifampin, and streptomycin.

Treatment of mycobacterial infection in an immunocompromised patient

Another embodiment of the invention involves treating an immunocompromised patient infected by a prokaryote of the genus Mycobacterium by administering to the patient a therapeutically effective amount of a neutral 3:1 gallium complex of a 3-hydroxy-4-pyrone. The neutral 3:1 (hydroxypyrone:gallium) complex is one in which the hydroxypyrone is selected from the group of compounds represented by Formula 1, as defined above. The therapeutically effective amount is that which provides a blood plasma gallium concentration that is sufficient to facilitate the antimicrobial combination therapy, while not creating any additional complications in the immunocompromised patent.

Of particular interest is treatment of those patients that have been infected with species such as M. tuberculosis and M. leprae, as well as species such as M. avium, M. aurum, and M. smegmatis, which may pose a health risk to immunocompromised patients.

In an exemplary dosing regimen, an immunocompromised patient infected by a pathogenic species of Mycobacterium will be given about 100 to 750 mg/day of the complex, for about 30 to 365 days, or longer. In addition, it may be desired to include additional therapeutic agents with the neutral 3:1 gallium complex of a 3-hydroxy-4-pyrone in the treatment of an immunocompromised patient. Such additional agents include, by way of example and not limitation, one or more antimicrobial agents listed previously herein.

The complex may also be co-administered with other therapeutic agents that the immunocompromised patient may already be taking. These include, by way of illustration and not limitation, antiretroviral agents, particularly those used in AIDS therapy, including without limitation nucleoside analogs such as zidovudine (AZT), ddI and ddC; protease inhibitors such as saquinavir, ritonavir, indinavir, and nelfinavir; and non-nucleoside reverse transcriptase inhibitors such as nevirapine and delavirdine; and so forth. In considering combination therapy, it is important to evaluate how the various therapeutic agents might interact. Many of the aforementioned drugs are nucleoside analogs, which inhibit polymerization of DNA as it is replicated. Gallium is expected to work synergistically with these nucleoside analogs. Gallium inhibits ribonucleotide reductase, and thus inhibits the production of the nucleosides required for DNA synthesis. As a result, the relative proportion of nucleoside analogs to native nucleosides will increase, which will further inhibit bacterial and retroviral DNA synthesis.

The treatment of an immunocompromised patient with a gallium complex containing a 3-hydroxy-4-pyrone is not limited to those patients whose immunodeficiency is associated with retroviral infection (such as patients with HIV infection). The gallium complexes of the present invention can also be effectively administered to other classes of patients with an immunocompromised status, such as those undergoing organ transplant or those suffering from an immunodeficiency of genetic origin.

An acknowledged advantage of combination therapy is that it reduces the emergence of resistant strains, due to the low probability of a single organism simultaneously acquiring multiple mutations that would confer resistance to each of the administered agents. The greater the structural and mechanistic differences between the combined agents, the lower the likelihood that simultaneous multiple resistance-conferring mutations will arise. As a primary mechanism of gallium action is novel (the disruption of bacterial iron uptake and metabolism), the addition or substitution of gallium to an existing combination therapy regimen therefore reduces the probability that drug resistance will develop.

Prevention of Mycobacterial Infection

Another embodiment of the invention involves the prophylactic treatment of a patient to prevent infection by a prokaryote of the genus Mycobacterium by administering to the patient a prophylactically effective amount of a neutral 3:1 gallium complex of a 3-hydroxy-4pyrone. The neutral 3:1 (hydroxypyrone:gallium) complex is one in which the hydroxypyrone is selected from the group of compounds represented by of Formula 1, as defined above. The therapeutically effective amount is such that a blood plasma gallium concentration is provided that is sufficient to enable prevention of the infection.

Of particular interest is the prevention of infection with Mycobacterium tuberculosis. The method of the invention can be used to administer a neutral 3:1 gallium complex of a 3-hydroxy-4-pyrone to uninfected individuals, including but not restricted to patients that are known to have been exposed to M. tuberculosis or to individuals or patients who may come in contact with infected individuals, such as social workers, health care professionals, and so forth.

In an exemplary dosing regimen, a prophylactically effective dose will be about 20 to 500 mg/day of the complex, for about 30 to 180 days, which is the time frame needed for effective prophylaxis. In another exemplary dosing regimen, a preventative dosage against a mycobacterial species is about 150 to 750 mg/day of the complex, for about 30 to 180 days.

It is to be understood that while the invention has been described in conjunction with the preferred specific embodiments thereof, the foregoing description, as well as the examples that follow, are intended to illustrate and not limit the scope of the invention. Other aspects, advantages, and modifications will be apparent to those skilled in the art to which the invention pertains.

All patents, patent documents, and publications cited herein are hereby incorporated by reference in their entirety for their disclosure concerning any pertinent information not explicitly included herein.

### EXAMPLES

The following examples are put forth so as to provide those of ordinary skill in the art with a complete disclosure and description of how to make and use the compounds of this invention, and are not intended to limit the scope of what the inventor regards as his invention. These examples focus primarily on gallium maltolate or gallium ethyl maltolate as representative complexes of a 3-hydroxy-4-pyrone with gallium as claimed in the present invention and should not be regarded as restrictive with respect to the preferred choice of a 3-hydroxy-4-pyrone. Efforts have been made to ensure accuracy with respect to numbers (e.g., amounts, temperature, etc.) but some errors and deviations should be accounted for. Unless otherwise indicated, parts are parts by weight, temperature is in degrees Celsius, and pressure is at or near atmospheric. All solvents were purchased as HPLC or reagent grade and, where appropriate, solvents and reagents were analyzed for purity using common techniques. Also, in the x-ray fluorescence and diffraction data given in Examples 1 and 2, the numbers in parentheses after the value reported represent the estimated standard deviation in the last digit(s).

### Example 1

### Preparation of Gallium Ethyl Maltolate

A 1.5M solution of ethyl maltol in chloroform was mixed with an equal volume of a 0.5M solution of gallium nitrate nonohydrate in ethanol to provide a 3:1 molar ratio of ethyl maltol to gallium ions in the mixture. The mixture was stirred for 7 minutes at 22° C. Solid anhydrous sodium carbonate was then added in a 10 molar excess, and stirring continued for an additional ten minutes. When the sodium carbonate was added, a trace of water was added to facilitate the reaction. The mixture was then filtered and the filtrate evaporated to give the solid 3:1 complex of ethyl maltol and gallium. The complex as so produced contained 14.3(1) wt % gallium by x-ray fluorescence analysis, as predicted for Ga(C₇H₆O₃)₃. The complex formed white to pale beige monoclinic crystals with unit cell parameters of about a=7.899(1)Å, b=8.765(1)Å, c=31.626(2)Å, beta—103.253(7) degrees, V=2131 Å³, based on powder x-ray diffraction analysis. The solubility of this compound was measured as about 5 millimolar in distilled deionized water at 23° C.

### Example 2

### Preparation of Gallium Maltolate

Maltol was dissolved in chloroform to form a 0.75M solution, and gallium nitrate nonohydrate was dissolved in ethanol to form a 0.5M solution. To 20 mL of the 0.75M maltol solution in chloroform was slowly added, with continuous stirring, 10 mL of the 0.5M gallium nitrate nonohydrate solution in ethanol. The resulting solution was stirred for 5 minutes at 23° C. About 5.5 grams of powdered anhydrous sodium carbonate were added, and stirring continued for an additional 12 minutes. The mixture was filtered to remove all solids, and the filtrate was evaporated in a rotary evaporator. The remaining crystalline solid was the 3:1 maltol:gallium complex. This complex was analyzed using powder x-ray diffraction and found to consist of orthorhombic crystals with unit cell dimensions of about a=8.52(1)Å, b=16.94(1)Å, c=12.02(1)Å. The solubility of this composition was measured as about 24 millimolar in distilled deionized water at 23° C.

### Example 3

### Treatment with Oral Gallium Maltolate of Guinea Pigs Infected with Mycobacterium tuberculosis

The efficacy of gallium maltolate in the treatment of M. tuberculosis infection was studied using male Hartley guinea pigs (average weight 700 mg). Twelve animals received an aerosol dose of 1.1×10⁵ CFU (colony forming units) of the Erdman strain of M. tuberculosis (the same organism that infects humans). This dose resulted in the implantation of 75-100 bacteria in the lungs. Each animal was housed in its own cage and was supplied with HEPA-filtered air. Seven days after infection, the twelve guinea pigs were divided into four equal groups. Each group received a different daily dose of gallium maltolate: 0 (control), 3.3 mg/Kg, 10 mg/Kg, and 30 mg/Kg. The appropriate amounts of gallium maltolate were dissolved in 5 mL of deionized, ultrafiltered water and the resulting solutions were administered manually to the guinea pigs by oral gavage. The weight of each animal was measured each day. Twenty-one days after infection, the twelve guinea pigs were sacrificed. Necropsies were performed to assess the extent of disease. The lungs, livers, and spleens in particular were observed. The spleens were weighed and tissue samples taken for bacterial culturing.

All of the gallium maltolate-treated animals had strikingly fewer tubercules (white, partially calcified nodules resulting from M. tuberculosis infection) in the lungs and livers relative to the untreated animals. In addition, cultures taken from the spleens showed a five-fold decrease in CFUs for the 3.3 mg/Kg dose group compared to the controls, while the 10 mg/Kg and 30 mg/Kg dose groups showed a ten-fold decrease. These decreases in CFUs are greater than those observed for ethambutol and slightly less than those observed for isoniazid. The results of this experiment show that orally administered gallium maltolate, even at low daily doses, is effective in treating M. tuberculosis infection in the guinea pig, which is an accepted and preferred animal model for studying the human disease.

### Example 4

### Preparation of Capsules Containing a Pharmaceutically Acceptable Buffer

The purpose of this example is to demonstrate the preparation of an orally deliverable pharmaceutical composition containing a neutral complex of gallium and a 3-hydroxy-4-pyrone, where the means to inhibit dissociation of the complex in the acidic conditions of the stomach is the use of a pharmaceutically acceptable buffer. Specifically, 50 mg of gallium maltolate, about 50 to about 1000 mg (preferably 500 mg) of calcium carbonate, and an amount of starch sufficient to complete filling of a standard gelatin capsule, are added to a standard gelatin capsule. The capsule is then closed to provide a composition of this invention. Such a capsule will inhibit the dissociation of the 3:1 maltol:gallium composition (gallium maltolate) in the acidic conditions of the stomach.

In view of the above, other neutral complexes of gallium and 3-hydroxy-4-pyrones could be prepared using the methods described above by merely substituting such other 3-hydroxy-4-pyrones for maltol. Similarly, other means to prevent dissociation of the neutral complex could be employed by merely substituting such other means for the means exemplified above.

Specifically, from about 50 to about 1000 mg of other pharmaceutically acceptable buffers or salts can be employed in place of calcium carbonate. Such other pharmaceutically acceptably buffers or salts include, by way of example, sodium bicarbonate, sodium carbonate, and the like.

### Example 5

### Clinical Evaluation of Gallium Maltolate for Treating Pulmonary M. tuberculosis Infection in Combination with Known Therapies

Gallium maltolate is evaluated clinically for efficacy in treating M. tuberculosis infection. Adult patients with sputum smear-positive pulmonary tuberculosis are selected for the study. The methods employed in this clinical study would be those described in Kennedy et al., “Randomized controlled trial of a drug regimen that includes ciprofloxacin for the treatment of pulmonary tuberculosis,” Clin. Infect. Dis. 22(5):827-33 (1996).

Patients with M. tuberculosis infection are treated once per day with known therapies along with gelatin capsules containing gallium maltolate. Patients are divided randomly and blindly into two approximately equal groups (Group 1 and Group 2). These two groups are each then subdivided into four approximately equal subgroups that receive either 0 (placebo), 125, 250, or 500 mg/day gallium maltolate for about six months. The patients in Group 1 receive the gallium maltolate dose in addition to a combination anti-tuberculosis therapy of: 300 mg/day isoniazid, 600 mg/day rifampin, and 15 mg/Kg/day ethambutol. The patients in Group 2 receive the gallium maltolate dose in addition to a combination anti-tuberculosis therapy of: 300 mg/day isoniazid and 750 mg/day ciprofloxacin. Patients are monitored for the bacteriological presence of M. tuberculosis in sputum smears and cultures. Patients are also medically monitored for M. tuberculosis disease, including examination for medical symptoms of M. tuberculosis infection such as possible detection of pulmonary cavitation and/or calcification by x-ray examinations throughout the study. The sputum smears and cultures and medical examination are done after the first week, the second week, and every two weeks thereafter. In addition, lung histopathology is studied using biopsy, immunostaining and bacteriological culturing, with lung biopsy samples obtained from the patients at 0, 30, 90, and 180 days following initiation of therapy, sampling in regions showing, or previously showing, radiologic evidence of tuberculosis.

Experimental clinical work conducted according to the aforementioned procedures can be used to evaluate the efficacy of gallium maltolate and related compounds of this invention for treating M. tuberculosis infection in combination with known therapies. It is expected that the gallium complexes of the invention will be shown to be effective additions to any and all combination therapy regimes, including the two combination therapies described in the preceding paragraph, with no adverse effects attributable to the addition of these agents to either regime. Addition of gallium in particular is expected to shorten the course of treatment; such a shortening of treatment should increase patient compliance and thus further decrease the likelihood that drug resistance will emerge.

### Example 6

### Preparation of Enteric Coated Capsule Formulation

The purpose of this example is to demonstrate the preparation of an orally deliverable pharmaceutical composition containing a neutral complex of gallium and a 3-hydroxy-4-pyrone, where the means to inhibit dissociation of the complex in the acidic conditions of the stomach is the use of an enteric coating. Enteric coating of a gallium:3-hydroxy-4-pyrone complex is anticipated to retard or inhibit release of the complex in the acidic conditions of the stomach and allow the complex to be specifically released into the contents of the intestine and distal to the stomach. Specifically, into a standard size 3 hard gelatin capsule (about 15.5 mm long and 5.8 mm diameter) is added 40 mg of a 3:1 maltol:gallium composition, 10 mg of maltol, and about 190 mg of starch. The capsule is closed and is then coated with a layer of cellulose acetate phthalate/diethyl phthalate using a pilot-scale procedure described by Jones, Manufacturing Chemist & Aerosol News 41:43-57 (1970). Acetone is used as a solvent, and a coating thickness of about 35 micrometers is obtained. Such a capsule inhibits the release of its contents (the 3:1 maltol:gallium composition) in the acidic conditions of the stomach, but releases its contents in the small intestine, where the pH is greater than about 5.5. Other materials well known in the art can be used to enterically coat the capsule by merely substituting another suitable material for the cellulose acetate phthalate/diethyl phthalate employed above. Such other materials include, by way of example, cellulose acetate phthalate, hydroxypropyl methylcellulose phthalate, poly(vinyl acetate phthalate), hydroxypropyl methylcelluloseacetate succinates, poly(meth)acrylates, and the like.

### Example 7

### Clinical Evaluation of Gallium Maltolate for Preventing M. tuberculosis Infection in HIV Infected Persons

Gallium maltolate is evaluated clinically for efficacy in treating M. tuberculosis infection in HIV infected patients. The clinical methods employed in this clinical study are described in Gordin et al., “Rifampin and pyrazinamide vs. isoniazid for prevention of tuberculosis in HIV-infected persons: an international randomized trial,” JAMA 238(11): 1445-50 (2000). Patients with HIV infection are treated once per day with enteric-coated capsules containing gallium maltolate. For HIV disease solely, the patients are also given 300 mg AZT twice per day, 200 mg ddI twice per day, and 800 mg indinavir every eight hours. The patients are selected for TB-negative status by skin test and lung x-ray status, and sputum smear and cultures. Patients are divided randomly and blindly into approximately six equal groups, with the first four groups receiving: Group 1: 0 (placebo); Group 2: 250; Group 3: 500; and Group 4: 750 mg/day gallium maltolate for about six months. These first four groups of patients are compared to the last two groups of HIV patients who receive: Group 5: isoniazid, 300 mg/d with pyridoxine hydrochloride (B vitamin supplement); Group 6: rifampin, 600 mg/d and pyrazinamide 20 mg/kg per day. Patients are medically monitored for symptoms of HIV infection and for new M. tuberculosis infection throughout the study. In addition, blood serum samples are obtained from the patients at days 0, 30, 90, and 180 and are assayed for CD4 and CD8 T-cell counts by routine methods and for HIV-1 RNA (viral load assay) using the Roche AMPLICOR assay (Sun et al., J. Clin. Microbiol. 36(10):2964-2969 (1998)). The primary end point for M. tuberculosis pathogenesis is culture-confirmed tuberculosis, with a secondary end point being proven or probable tuberculosis.

Experimental work conducted according to the aforementioned procedures can be used to evaluate the efficacy of gallium maltolate and related compounds of this invention for both treating HIV infection and preventing M. tuberculosis infection. It is expected that patients in Group 2, Group 3, Group 4, Group 6, Group 7, and Group 8 will be statistically less likely to have tuberculosis in conjunction with increased CD4 T cell counts compared to Group 1 and Group 5.

### Example 8

### Clinical Evaluation of Gallium Maltolate for Treating M. leprae Infection in Combination with Known Therapies

Gallium maltolate is evaluated clinically for efficacy in treating M. leprae infection. Adult patients with nasal smear-positive leprosy infections are selected for the study. Multibacillary (or lepromatous) leprosy is characterized by large numbers of organisms, and is generally difficult to treat because of the high likelihood that drug-resistant organisms will emerge (Katzung, Basic and Clinical Pharmacology, 1998 Apppleton & Lange, Simon & Schuster). The standard regimen of multidrug therapy for multibacillary Hansen's disease (leprosy) typically lasts 24 months and employs three agents: dapsone (100 mg), clofazimine (50 mg) (both administered daily) and rifampin (600 mg) (administered monthly with an additional dose of clofazimine (300 mg)).

The methods employed in this clinical study are described by Baohong et al., “Bactericidal Activity of Single Dose of Clarithromycin plus Minocycline, with or without Ofloxacin, against Mycobacterium leprae in Patients,” Antimicrob Agents Chemother 40(9):2137-41 (1996). The efficacy of supplementing treatment for patients with M. leprae infection by treatment once per day with enteric coated capsules containing gallium maltolate is evaluated. Patients are divided randomly and blindly into two groups, Group 1 and Group 2. Each of these two groups is then subdivided into four approximately equal subgroups, which receive either 0 (placebo), 250, 500, or 750 mg/day gallium maltolate for the first 12 months of infection therapy. Group 1 receives the gallium complex as indicated, in addition to the traditional combination anti-leprosy therapy of: 100 mg/day dapsone; 50 mg/day clofazimine; 600 mg rifampin once monthly together with an additional dose of 300 mg clofazimine. Group 2 also receives the gallium complex as indicated, in addition to the alternative combination anti-leprosy therapy of: a monthly dose of 600 mg rifampin, 1600 mg clarithromycin, 160 mg minocycline, and 650 mg ofloxacin. All patients are monitored for the bacteriological presence of M. leprae in nasal smears and cultures. Patients are also medically monitored for M. leprae disease, including: examination for medical symptoms of M. leprae infection, including cutaneous and neural manifestations of disease; histopathologic examination of biopsy tissue; and “culturing” of all identified lesions by mouse footpad inoculation (Dhople et al., Indian J. Lepr. 63(2):166-79 (1991); and Dhople et al., Arzneimittelforschung 41(3):253-56 (1991)). The smears, cultures, and medical examinations are performed after the first week, the second week, and every two weeks thereafter. In addition, cutaneous lesion biopsy samples are obtained from the patients at 0, 30, 90, and 180 days, and every 90 days thereafter. Lesion histopathology is studied using immunostaining and bacteriological culturing.

Experimental work conducted according to the aforementioned procedures can be used to evaluate the efficacy of gallium maltolate and related compounds of this invention for treating M. leprae infection. It is expected that the gallium complexes of the invention will be effective additions to one or both of the studied combination therapy regimes, with no adverse effects attributable to the addition of these agents to either regime. Addition of gallium is expected to shorten the course of treatment in both regimes.

I claim:

1. A method of treating a patient infected by a prokaryote of the genus Mycobacterium by administering a therapeutically effective amount of a neutral 3:1 gallium complex of a 3-hydroxy-4-pyrone, where the 3-hydroxy-4-pyrone has the formula: 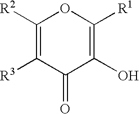
wherein R¹, R², and R³ are independently selected from the group consisting of hydrogen and a —C₁₋₆ alkyl group.

2. The method of claim 1 wherein the complex is administered in a pharmaceutical composition containing a pharmaceutically acceptable carrier.

3. The method of claim 1 wherein the blood plasma gallium concentration obtained is in the range of approximately 1 to 5000 ng/mL.

4. The method of claim 3 wherein the blood plasma gallium concentration obtained is in the range of approximately 100 to 1500 ng/mL.

5. The method of claim 1 wherein the complex is administered orally.

6. The method of claim 5 wherein the complex is administered in a dose of approximately 10 to 2500 mg per day.

7. The method of claim 6 wherein the complex is administered in a dose of approximately 150 to 750 mg per day.

8. The method of claim 7 wherein the complex is administered for about 30 to 365 days.

9. The method of claim 8 wherein the complex is administered for about 30 to 180 days.

10. The method of claim 2 wherein the pharmaceutically acceptable carrier is suitable for oral administration.

11. The method of claim 10 wherein the carrier is a solid.

12. The method of claim 11 wherein the pharmaceutical composition is in the form of a tablet or capsule.

13. The method of claim 10 wherein the pharmaceutical composition is encapsulated in a material that does not dissolve until the small intestine of the individual is reached.

14. The method of claim 10 wherein the carrier is a liquid.

15. The method of claim 1 wherein R¹, R², and R³ are H.

16. The method of claim 1 wherein R¹ is —CH₃; and R² and R³ are H.

17. The method of claim 1 wherein R¹ is —C₂H₅; and R² and R³ are H.

18. The method of claim 1 wherein R² is —CH₃; and R² and R³ are H.

19. The method of claim 2 wherein the pharmaceutical composition further includes at least one antimicrobial agent selected from the group consisting of amikacin, aminosalicylic acid, azithromycin, capreomycin, ciprofloxacin, clarithromycin, clofazimine, cycloserine, dapsone, erythromycin, ethambutol, ethionamide, isoniazid, kanamycin, minocycline, ofloxacin, protionamide, pyrazinamide, rifabutin, rifampicine, rifampin, sparfloxacin, streptomycin, trimethoprim sulfamethoxazole, tobramycin, and viomycin, and combinations thereof.

20. The method of claim 19 wherein the pharmaceutical composition includes one or more agents selected from the group consisting of ethambutol, isoniazid, pyrazinamide, rifampin, and streptomycin.

21. The method of claim 1 wherein the Mycobacterium is selected from the group consisting of M. africanum, M. aurum, M. avium, M. avium paratuberculosis, M. balnei, M. bovis, M. chelonae, M. genitalium, M. gallisepticum, M. gastri, M. goodii, M. gordonae, M. haemophilum, M. intracellulare, M. kansasii, M. leprae, M. lepraemurium, M. malmoense, M. microti, M. penetrans, M. platypoecilus, M. pneumoniae, M. scrofulvaeum, M. simiae, M. smegmatis, M. szulgai, M. terrae-trivial, M. tuberculosis, M. ulcerans, and M. xenopi.

22. The method of claim 21 wherein the Mycobacterium is M. tuberculosis.

23. The method of claim 22 wherein about 150 to 750 mg/day of the complex is administered for about 30 to 180 days.

24. The method of claim 21 wherein the Mycobacterium is M. leprae.

25. The method of claim 24 wherein about 150 to 750 mg/day of the complex is administered for about 30 to 365 days.

26. An improved method of treating a patient infected by a prokaryote of the genus Mycobacterium by administering to the patient a combination of antimicrobial agents selected from the group consisting of amikacin, aminosalicylic acid, azithromycin, capreomycin, ciprofloxacin, clarithromycin, clofazimine, cycloserine, dapsone, erythromycin, ethambutol, ethionamide, isoniazid, kanamycin, minocycline, ofloxacin, protionamide, pyrazinamide, rifabutin, rifampicine, rifampin, sparfloxacin, streptomycin, trimethoprim sulfamethoxazole, tobramycin, and viomycin; the improvement comprising administering a therapeutically effective amount of a neutral 3:1 gallium complex of a 3-hydroxy-4-pyrone, where the 3-hydroxy-4-pyrone has the formula: 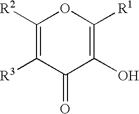
wherein R¹, R², and R³ are independently selected from the group consisting of hydrogen and a —C₁₋₆alkyl group.

27. The method of claim 26 wherein the complex is administered in a pharmaceutical composition containing a pharmaceutically acceptable carrier.

28. The method of claim 26 wherein the blood plasma gallium concentration obtained is in the range of approximately 1 to 5000 ng/mL.

29. The method of claim 28 wherein the blood plasma gallium concentration obtained is in the range of approximately 100 to 1500 ng/mL.

30. The method of claim 26 wherein the complex is administered orally.

31. The method of claim 30 wherein the complex is administered in a dose of approximately 10 to 2500 mg per day.

32. The method of claim 31 wherein the complex is administered in a dose of approximately 150 to 750 mg per day.

33. The method of claim 32 wherein the complex is administered for about 30 to 365 days.

34. The method of claim 33 wherein the complex is administered for about 30 to 180 days.

35. The method of claim 27 wherein the pharmaceutically acceptable carrier is suitable for oral administration.

36. The method of claim 35 wherein the carrier is a solid.

37. The method of claim 36 wherein the pharmaceutical composition is in the form of a tablet or capsule.

38. The method of claim 35 wherein the pharmaceutical composition is encapsulated in a material that does not dissolve until the small intestine of the individual is reached.

39. The method of claim 35 wherein the carrier is a liquid.

40. The method of claim 26 wherein R¹, R², and R³ are H.

41. The method of claim 26 wherein R¹ is —CH₃; and R² and R³ are H.

42. The method of claim 26 wherein R¹ is —C₂H₅; and R² and R³ are H.

43. The method of claim 26 wherein R² is —CH₃; and R¹ and R³ are H.

44. The method of claim 26 wherein the Mycobacterium is selected from the group consisting of M. africanum, M. aurum, M. avium, M. avium paratuberculosis, M. balnei, M. bovis, M. chelonae, M. genitalium, M. gallisepticum, M. gastri, M. goodii, M. gordonae, M. haemophilum, M. intracellulare, M. kansasii, M. leprae, M. lepraemurium, M. malmoense, M. microti, M. penetrans, M. platypoecilus, M. pneumoniae, M. scrofulvaeum, M. simiae, M. smegmatis, M. szulgai, M. terrae-trivial, M. tuberculosis, M. ulcerans, and M. xenopi.

45. The method of claim 44 wherein the Mycobacterium is M. tuberculosis.

46. The method of claim 45 wherein about 150 to 750 mg/day of the complex is administered for about 30 to 180 days.

47. The method of claim 44 wherein the Mycobacterium is M. leprae.

48. The method of claim 47 wherein about 150 to 750 mg/day of the complex is administered for about 30 to 365 days.

49. The method of claim 26 wherein the combination of antimicrobial agents comprises three or more antimicrobial agents selected from the group consisting of ethambutol, isoniazid, pyrazinamide, rifampin, and streptomycin.

50. The method of claim 26, which further comprises administering one or more nucleoside analogs.

51. The method of claim 50 wherein the nucleoside analogs are selected from the group consisting of AZT, ddI, ddC, acyclovir, gancyclovir, and foscarnet.

52. The method of claim 26, which further comprises administering one or more protease inhibitors.

53. The method of claim 52 wherein the protease inhibitors are selected from the group consisting of saquinavir, ritonavir, indinavir, and nelfinavir.

54. The method of claim 26, which further comprises administering one or more non-nucleoside reverse transcriptase inhibitors.

55. The method of claim 54 wherein the reverse transcriptase inhibitors are selected from the group consisting of nevirapine and delavirdine.

56. A method of treating an immunocompromised patient infected by a prokaryote of the genus Mycobacterium by administering a therapeutically effective amount of a neutral 3:1 gallium complex of a 3-hydroxy-4-pyrone, where the 3-hydroxy-4-pyrone has the formula: 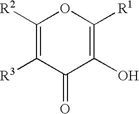
wherein R¹, R², and R³ are independently selected from the group consisting of hydrogen and a —C₁₋₆alkyl group.

57. The method of claim 56 wherein the complex is administered in a pharmaceutical composition containing a pharmaceutically acceptable carrier.

58. The method of claim 56 wherein the blood plasma gallium concentration obtained is in the range of approximately 1 to 5000 ng/mL.

59. The method of claim 58 wherein the blood plasma gallium concentration obtained is in the range of approximately 100 to 1500 ng/mL.

60. The method of claim 56 wherein the complex is administered orally.

61. The method of claim 60 wherein the complex is administered in a dose of approximately 10 to 2500 mg per day.

62. The method of claim 61 wherein the complex is administered in a dose of approximately 150 to 750 mg per day.

63. The method of claim 62 wherein the complex is administered for about 30 to 180 days.

64. The method of claim 63 wherein the complex is administered for about 30 to 365 days.

65. The method of claim 57 wherein the pharmaceutically acceptable carrier is suitable for oral administration.

66. The method of claim 65 wherein the carrier is a solid.

67. The method of claim 66 wherein the pharmaceutical composition is in the form of a tablet or capsule.

68. The method of claim 65 wherein the pharmaceutical composition is encapsulated in a material that does not dissolve until the small intestine of the individual is reached.

69. The method of claim 65 wherein the carrier is a liquid.

70. The method of claim 56 wherein R¹, R², and R³ are H.

71. The method of claim 56 wherein R¹ is —CH₃; and R² and R³ are H.

72. The method of claim 56 wherein R¹ is —C₂H₅; and R¹ and R³ are H.

73. The method of claim 56 wherein R² is —CH₃; and R¹ and R³ are H.

74. The method of claim 57 wherein the pharmaceutical composition further includes at least one antimicrobial agent selected from the group consisting of amikacin, aminosalicylic acid, azithromycin, capreomycin, ciprofloxacin, clarithromycin, clofazimine, cycloserine, dapsone, erythromycin, ethambutol, ethionamide, isoniazid, kanamycin, minocycline, ofloxacin, protionamide, pyrazinamide, rifabutin, rifampicine, rifampin, sparfloxacin, streptomycin, trimethoprim sulfamethoxazole, tobramycin, and viomycin, and combinations thereof.

75. The method of claim 74 wherein the pharmaceutical composition includes three or more agents selected from the group consisting of isoniazid, rifampin, pyrazinamide, streptomycin, and ethambutol.

76. The method of claim 56 wherein the Mycobacterium is selected from the group consisting of M. africanum, M. aurum, M. avium, M. avium paratuberculosis, M. balnei, M. bovis, M. chelonae, M. genitalium, M. gallisepticum, M. gastri, M. goodii, M. gordonae, M. haemophilum, M. intracellulare, M. kansasii, M. leprae, M. lepraemurium, M. malmoense, M. microti, M. penetrans, M. platypoecilus, M. pneumoniae, M. scrofulvaeum, M. simiae, M. smegmatis, M. szulgai, M. terrae-trivial, M. tuberculosis, M. ulcerans, and M. xenopi.

77. The method of claim 76 wherein the Mycobacterium is M. tuberculosis.

78. The method of claim 77 wherein about 150 to 750 mg/day of the complex is administered for about 30 to 180 days.

79. The method of claim 76 wherein the Mycobacterium is M. leprae.

80. The method of claim 79 wherein about 150 to 750 mg/day of the complex is administered for about 30 to 365 days.

81. The method of claim 56, which further comprises administering one or more nucleoside analogs.

82. The method of claim 81 wherein the nucleoside analogs are selected from the group consisting of AZT, ddI, ddC, acyclovir, gancyclovir, and foscarnet.

83. The method of claim 56, which further comprises administering one or more protease inhibitors.

84. The method of claim 83 wherein the protease inhibitors are selected from the group consisting of saquinavir, ritonavir, indinavir, and nelfinavir.

85. The method of claim 56, which further comprises administering one or more non-nucleoside reverse transcriptase inhibitors.

86. The method of claim 85 wherein the reverse transcriptase inhibitors are selected from the group consisting of nevirapine and delavirdine.

87. A method of preventing infection by a prokaryote of the genus Mycobacteria by administering a prophylactically effective amount of a neutral 3:1 gallium complex of a 3-hydroxy-4-pyrone, where the 3-hydroxy-4-pyrone has the formula: 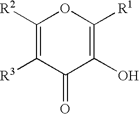
wherein R¹, R² and R³ are independently selected from the group consisting of hydrogen and a —C₁₋₆alkyl group.

88. The method of claim 87 wherein the complex is administered in a pharmaceutical composition containing a pharmaceutically acceptable carrier.

89. The method of claim 87 wherein the blood plasma gallium concentration obtained is in the range of approximately 1 to 5000 ng/mL.

90. The method of claim 89 wherein the blood plasma gallium concentration obtained is in the range of approximately 50 to 1000 ng/mL.

91. The method of claim 87 wherein the complex is administered orally.

92. The method of claim 91 wherein the complex is administered in a dose of approximately 10 to 2500 mg per day.

93. The method of claim 92 wherein the complex is administered in a dose of approximately 20 to 500 mg per day.

94. The method of claim 93 wherein the complex is administered for about 30 to 180 days.

95. The method of claim 92 wherein the complex is administered in a dose of approximately 150 to 750 mg per day.

96. The method of claim 95 wherein the complex is administered for about 30 to 180 days.

97. The method of claim 88 wherein the pharmaceutically acceptable carrier is suitable for oral administration.

98. The method of claim 97 wherein the carrier is a solid.

99. The method of claim 98 wherein the pharmaceutical composition is in the form of a tablet or capsule.

100. The method of claim 97 wherein the pharmaceutical composition is encapsulated in a material that does not dissolve until the small intestine of the individual is reached.

101. The method of claim 97 wherein the carrier is a liquid.

102. The method of claim 87 wherein R¹, R², and R³ are H.

103. The method of claim 87 wherein R¹ is —CH₃; and R² and R³ are H.

104. The method of claim 87 wherein R¹ is —C₂H₅; and R² and R³ are H.

105. The method of claim 87 wherein R² is —CH₃; and R² and R³ are H.

106. The method of claim 87 wherein the Mycobacterium is selected from the group consisting of M. africanum, M. aurum, M. avium, M. avium paratuberculosis, M. balnei, M. bovis, M. chelonae, M. genitalium, M. gallisepticum, M. gastri, M. goodii, M. gordonae, M. haemophilum, M. intracellulare, M. kansasii, M. leprae, M. lepraemurium, M. malmoense, M. microti, M. penetrans, M. platypoecilus, M. pneumoniae, M. scrofulvaeum, M. simiae, M. smegmatis, M. szulgai, M. terrae-trivial, M. tuberculosis, M. ulcerans, and M. xenopi.

107. The method of claim 106 wherein the Mycobacterium is M. tuberculosis.

108. The method of claim 106 wherein the Mycobacterium is M. leprae.
